(19) United States
(12) Patent Application Publication
(10) Pub. No.: US 2025/0256046 A1
(43) Pub. Date: Aug. 14, 2025

(54) MEDICAL DEVICE

(71) Applicant: Drägerwerk AG & Co. KGaA, Lübeck (DE)

(72) Inventor: Markus HAMPE, Lübeck (DE)

(21) Appl. No.: 19/046,625

(22) Filed: Feb. 6, 2025

(30) Foreign Application Priority Data
Feb. 9, 2024 (DE) 10 2024 103 650.8

(51) Int. Cl. A61M 16/00

(52) U.S. Cl. CPC A61M 16/0003

(57) ABSTRACT

A medical device includes a base unit, a housing unit connectable to the base unit, a user interface connectable to the base unit, a number of connection units for reversibly connecting the base unit to the housing unit and for reversibly connecting the base unit to the user interface, and a coupling device. The coupling device is configured to change the number of connection units from a release state to a connection state and from the connection state to the release state.

### CROSS REFERENCE TO RELATED APPLICATIONS

This application claims the benefit of priority under 35 U.S.C. § 119 of German Application 10 2024 103 650.8, filed Feb. 9, 2024, the entire contents of which are incorporated herein by reference.

### TECHNICAL FIELD

The present invention pertains to a medical device comprising a base unit, a housing unit connectable to the base unit and a user interface connectable to the base unit.

### BACKGROUND

The possibility of combining a base unit with different user interfaces and different housing units means that the medical device can be adapted to different requirements.

DE 10 2018 005 280 B3 discloses a ventilator as an example of a medical device comprising a base unit with a cover plate, patient connections provided on the base unit and a control unit provided on the cover plate. This ventilator is configured so that the cover plate can be arranged on the base unit at different angles in relation to a vertical axis. For this purpose, the cover plate is connected to the base unit as part of a user interface via a plurality of components in a rotatable and lockable manner.

A disadvantage of the ventilator known from DE 10 2018 005 280 B3 is that although the control unit can be pivoted around a vertical axis of the base unit, it cannot be removed without disassembly. The accessibility of this ventilator is therefore disadvantageously limited.

### SUMMARY

It is therefore an object of the present invention to provide a medical device with which both a housing unit and a user interface can be securely connected to and disconnected from a base unit in a particularly simple manner, for example during device assembly or servicing.

These and other objects are attained by features according to the invention.

Further advantages and features of the invention are indicated by the description, the claims and the figures.

According to the invention, a medical device is provided. The medical device comprises a base unit, a housing unit connectable to the base unit, a user interface connectable to the base unit, a number of connection units for reversibly connecting the base unit to the housing unit and for reversibly connecting the base unit to the user interface, and a coupling device. The coupling device is configured to change the number of connection units from a release state to a connection state and from the connection state to the release state.

Thus, a medical device can be provided that is configured to be reversibly connected to a housing unit and a user interface by means of a number of connection units provided on the device side. Consequently, a modularity of the medical device can be improved. By providing a coupling device according to the invention, the number of connection units provided on the device side can be set to a connection state for connection to the housing unit and the user interface or to a release state for release from the housing unit and the user interface. In this way, simple assembly or disassembly of the housing unit and user interface is possible, e.g. to simplify assembly and/or servicing of the medical device.

In this respect, the object of the invention supports the concept of a modular structure of a medical device family in that, for example, a pre-produced base unit can be provided with different user interfaces and possibly different housing units with little assembly effort in a downstream customer-specific manufacturing step (“customizing”). This makes it easy to create devices in different comfort and price classes from similar base units by varying the user interfaces, for example with regard to the size of a user interface and the adjustability of the user interface relative to the base unit, and possibly by varying the housing units.

Preferably, at least part of the number of connection units is provided on the device side, i.e. formed as an assembly of the medical device, in particular as an assembly of the base unit.

The medical device can, for example, be configured as a ventilator or anesthesia device and have corresponding assemblies and interfaces.

For example, if the medical device is configured as a ventilator or anesthesia device, it may have a blower for controlling and/or regulating a volume flow and/or a pressure of a gas or gas mixture intended for ventilation. Furthermore, for example, the medical device may have a gas connection for supplying ambient air and/or compressed air and/or oxygen to the medical device. The medical device may further comprise, for example, one or more patient interfaces, such as an inspiratory and expiratory line. The medical device may further comprise, for example, a control unit and/or a power supply (e.g. mains connection and/or battery and/or accumulator). The medical device may, in particular if it is configured as a ventilator or as an anesthesia device, have further pneumatic components such as lines and valves, e.g. proportional valves, check valves, solenoid valves and the like for influencing the gas flow from the medical device to the patient and from the patient to the environment or to the medical device.

The base unit is used to accommodate at least electronic components and preferably also pneumatic components of the medical device. The base unit can be configured as a device chassis or comprise a device chassis and other assemblies, e.g. attachments.

The user interface may be configured to input and/or output information to a user of the medical device, e.g. a practitioner. For example, the user interface may include a display such as a screen to display information. The user interface can comprise a control unit, for example input elements such as knobs, buttons, rotary switches and/or a display, in particular a touch screen, for entering information.

A release state is understood to be a state in which the housing unit and/or the user interface can be removed from the medical device. Accordingly, a connection state is understood to be a state in which the housing unit and/or the user interface cannot be removed from the medical device, in other words are locked.

According to the invention, the coupling device is a coupling device of the medical device, in other words provided on the device side, in still other words an assembly of the medical device, preferably an assembly of the base unit.

The housing unit, the user interface, the number of connection units and the coupling device can each have a multi-part or multi-component configuration.

The number of connection units can be single or multiple (the number of connection units is one connection unit or a plurality of connection units).

Preferably, the coupling device has a number of actuators (actuating elements) for actuating the coupling device, wherein the number of actuators cannot be actuated without tools.

In this way, the safety of the medical device can be improved, as unintentional actuation of the actuators can be prevented.

Preferably, the medical device also has an openable cover which is configured to conceal the number of actuators when closed.

The medical device can thus be improved in terms of hygienic configuration.

The cover can, for example, be configured as a housing segment, for example as a component of the housing unit, which is or can be attached to the base unit. The cover can, for example, be detachably connected to the base unit, i.e. removable from it, in order to open the cover by removing or detaching it, or be pivotably connected to it, e.g. by means of a hinge or joint, in order to open the cover by pivoting.

Preferably, the coupling device has a number of locking shafts, each of the number of locking shafts being rotatable about a respective central axis so that the coupling device can be actuated.

The provision of a respective locking shaft enables the simultaneous action of this respective locking shaft on a plurality of connecting units, in particular those provided on the device side, provided that these are coupled, i.e. in interaction. In this respect, it is preferable that each of the number of locking shafts can be brought into engagement with a plurality of connection units, in particular those provided on the device side. In this way, by actuating only one actuator of the number of actuators, it is possible to influence a plurality of connection units, in particular those provided on the device side, which improves the handling of the medical device in that the time required to disassemble/assemble the housing unit and/or the user interface can be shortened.

Preferably, part of the number of connection units is configured as a number of connection units provided on the device side for reversibly connecting the base unit to the housing unit and for reversibly connecting the base unit to the user interface. Further preferably, a (i.e. other) part of the number of connection units is configured as a number of connection units provided on the user interface side for reversibly connecting the user interface to the base unit, wherein the number of connection units provided on the user interface side for connecting the user interface to the base unit can be positively connected to the number of connection units provided on the device side.

In this way, the user interface can be easily assigned to the base unit.

In the context of the invention, a connection unit provided on the device side is understood to be a connection unit which is configured as part of the medical device, in particular as part of the base unit.

The number of connection units provided on the user interface side can be the same as or different from the number of connection units provided on the device side.

Preferably, the number of connection units provided on the device side is configured to enable the housing unit and the user interface to be removed or fitted along a common axis, preferably along a vertical axis of the base unit, in the release state.

In this way, the manageability of the medical device during assembly and/or service can be improved.

A vertical axis of the base unit is understood to be a vertically aligned axis of the base unit or medical device in relation to an orientation of a base unit or medical device installed as intended.

Preferably, the number of connection units provided on the device side is configured as a plurality. Furthermore, preferably, the plurality of connection units provided on the device side is distributed evenly, in particular rotationally symmetrically, about the common axis in order to provide a-preferably corresponding-plurality of arrangements of the user interface and/or the housing unit relative to the base unit.

In this way, the arrangement of the user interface and/or the housing unit relative to the base unit can be configured flexibly. For example, patient interfaces, which can be provided on the base unit, and the user interface can each be positioned ergonomically in this way (e.g. patient interfaces are arranged for the patient, user interface arranged for the clinician or doctor).

Preferably, each connection unit of the number of connection units provided on the user interface side has at least one connection recess. Further preferably, each locking shaft of the number of locking shafts has at least one section with a shaft recess. Further preferably, each locking shaft of the plurality of locking shafts is configured such that, by rotating the locking shaft, the at least one shaft recess can be brought into alignment with a connection recess of the plurality of connection recesses in order to provide the release state. Furthermore, each locking shaft of the number of locking shafts is preferably configured such that the shaft recess cannot be brought into alignment with the connection recess of the number of connection recesses by rotating the locking shaft in order to provide the connection state.

In this way, a particularly simple way of realizing the release state and the connection state can be provided.

A connection recess is a recess in a connection unit.

A shaft recess is a recess in a locking shaft.

Particularly preferably, each locking shaft of the number of locking shafts has at least two sections with a shaft recess. Furthermore, preferably, each locking shaft of the number of locking shafts is configured such that, by rotating the respective locking shaft, the at least two shaft recesses can be brought into alignment with two corresponding connection recesses of the number of connection recesses in order to provide the release state. Furthermore, preferably, each locking shaft of the number of locking shafts is configured such that, by rotating the respective locking shaft, the at least two shaft recesses cannot be brought into alignment with two corresponding connection recesses of the number of connection recesses in order to provide the connection state.

This makes it particularly easy for a locking shaft to act simultaneously on at least two connection units provided on the device side. The handling effort can thus be improved.

Preferably, at least part of the number of connection units provided on the user interface side has a contact surface for clamping the housing unit between the user interface and the base unit. Furthermore, preferably, the clamping mounting can be achieved by providing the connection state by rotating the number of locking shafts.

In this way, the housing unit can be clamped between the user interface and the base unit, whereby the clamping mounting can advantageously be achieved essentially simultaneously with the provision of the connection state, i.e. can be achieved without additional coupling actuation.

Preferably, at least one sealing element is arranged between some or all of the number of contact surfaces and the number of connection units provided on the device side, which is configured to exhibit a preload by producing the clamping mounting.

In this way, a sealing effect can be achieved between the housing unit and the base unit.

The sealing element can, for example, be configured as an elastic sealing ring or as a crimp rib on the housing unit. The housing unit can, for example, have a sealing flange for contact with the sealing element or for holding the sealing element.

Preferably, the housing unit has a number of connection units provided on the housing unit side for reversible connection of the housing unit to the base unit.

In this way, a coupling between the housing unit and the base unit can be specifically adapted.

Preferably, the number of connection units provided on the housing unit side is configured to be positively connected in the connection state to the number of connection units provided on the device side and/or positively connected to the number of connection units provided on the user interface side.

This makes it particularly easy to connect the base unit to the housing unit and/or the base unit to the user interface.

Preferably, the medical device also has a locking device which is configured to lock the coupling device in the connection state.

In this way, unintentional release of the coupling device, i.e. an unintentional change from the connection state to the release state, can be prevented.

Preferably, the locking device comprises a locking actuating unit for actuating the locking device, wherein the locking actuating unit requires an actuating action for actuation which is different from an actuating action for actuating the coupling device.

In this way, it can be ensured that the locking device is not actuated unintentionally during operation of the coupling device.

A locking actuation unit is understood to be a unit for actuating the locking device. An actuating action is understood to be an action required by a user or operator of the medical device in order to actuate an element of the medical device, in particular the locking actuation unit and the coupling device.

Preferably, the medical device also has a number of additional connection units provided on the device side for reversibly connecting the base unit to the housing unit without the user interface being connected to the base unit.

While it is preferred that the user interface and the base unit accommodate the housing unit therebetween in a force-fit and/or form-fit manner, this is not necessary. According to the above preferred embodiment of the medical device, it is also possible in this respect for the base unit and housing unit to be connectable to one another without the user interface having to be connected to the base unit to establish this connection. In this way, for example, a sub-unit comprising the base unit and the housing unit can be provided and additionally equipped with a user interface in a subsequent step. In this way, the modularity of the medical device can be improved.

It is preferred that the housing unit is configured as a housing cover. It is also preferred that the housing unit has an additional number of connection units provided on the housing element side for reversible connection of the housing unit to the base unit.

It is preferred that the additional number of connection units provided on the housing element side is configured as a plurality of additional connection units provided on the housing element side in such a way that not all of the plurality of additional connection units provided on the housing element side engage with the base unit at the same time when the housing unit is placed on the base unit.

Preferably, the user interface has a first electrical and/or signaling interface. Furthermore, the base unit preferably has a corresponding second electrical and/or signaling interface. Further preferably, the first interface and the second interface can be connected to establish an electrical and/or signaling connection.

By connecting the aforementioned interfaces, it is thus possible to obtain an electrical and/or signal connection between the base unit and the user interface.

Preferably, an orientation of the first interface relative to the user interface and/or an orientation of the second interface relative to the base unit is variable.

In this way, the orientation of the first interface and/or the second interface can be adapted to a changing orientation of the user interface relative to the base unit.

Preferably, the first interface is configured as a connector which is floatingly mounted in or on the user interface, whereby the connector is spring-loaded in order to provide a contact pressure on the connector in the direction of the second interface.

In this way, a mechanical connection between the connector and the second interface can be reliably achieved.

The connector can be configured as a plug or as a slot (socket), i.e. as a male or female part of the pairing of the first and second interface.

It is preferred that the user interface and/or the base unit and/or the housing unit has a centering device in order to align the first interface and the second interface with each other in at least one direction when the user interface is placed on the base unit.

These and other features and advantages of the invention are also evident from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective exploded view of an embodiment of a medical device according to the invention;

FIG. 2 is a detailed view of a coupling device according to the invention, a number of connection units provided on the device side and a number of connection units provided on the user interface side;

FIG. 3a is a broken-out sectional view of a connection unit provided on the device side, a connection unit provided on the user interface side, a connection unit provided on the housing unit side and a locking shaft with further elements in the release state;

FIG. 3b is the broken out sectional view according to FIG. 3a, in a transition from the release state to a connection state;

FIG. 3c is the broken out sectional view according to FIG. 3a and FIG. 3b in the connected state;

FIG. 4a is a rear view of the medical device according to FIG. 1 with detail views of the actuators;

FIG. 4b is a perspective side and rear view of the medical device according to FIG. 1 showing the assembled state with the cover open;

FIG. 5 is a detailed perspective exploded view of a locking shaft with a part of a locking device;

FIG. 6a is a broken out sectional side view of the locking shaft according to FIG. 5 in the installed and non-locked state;

FIG. 6b is a broken-out sectional front view of the locking shaft in the installed and non-locked state according to FIG. 6a;

FIG. 6c is the broken out sectional side view of the locking shaft according to FIG. 6a with an actuating tool in a transitional state between locked and unlocked state;

FIG. 6d is a broken-out sectional front view of the locking shaft in the transitional state between locked and unlocked state according to FIG. 6c;

FIG. 6e is a broken out sectional side view of the locking shaft according to FIGS. 6a and 6c in a locked state;

FIG. 6f is a broken-out sectional front view of the locking shaft in the locked state according to FIG. 6e;

FIG. 7 is a detailed perspective view of a connection unit arranged on the user interface side,

FIG. 8a is a perspective front view of the medical device according to FIG. 1;

FIG. 8b is a perspective front view of a further embodiment of a medical device according to the invention;

FIG. 8c is a perspective front view of a further embodiment of a medical device according to the invention;

FIG. 9 is a broken-out sectional view with additional connection units provided on the device side and with a detailed view;

FIG. 10a is a broken-out detailed view of a first interface and a second interface in the disconnected state; and

FIG. 10b is a broken-out detailed view of the first interface and the second interface in the connected state.

### DESCRIPTION OF PREFERRED EMBODIMENTS

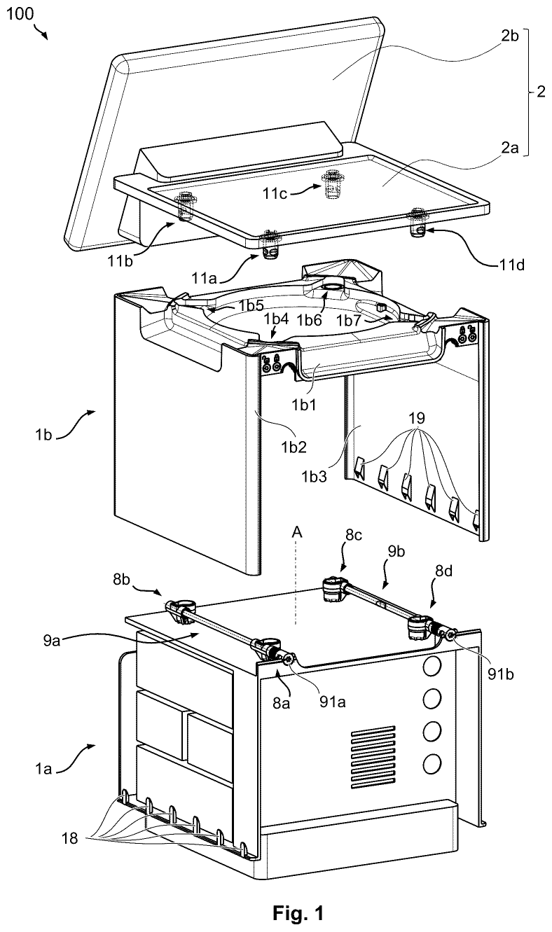

According to the invention, a medical device 100 is provided. An embodiment example of such a medical device 100 is shown in FIG. 1 in a perspective, rear exploded view.

A medical device 100 according to the invention comprises a base unit 1a, a housing unit 1b connectable to the base unit 1a, a user interface 2 connectable to the base unit 1a, a number of connection units 1b4, 1b5, 1b6, 1b7, 8a, 8b, 8c, 8d, 9a1, 9b1, 11a, 11b, 11c, 11d, 16, 19 for reversibly connecting the base unit 1a to the housing unit 1b and for reversibly connecting the base unit 1a to the user interface 2 as well as a coupling device 9a, 9b, which is configured to change the number of connection units 1b4, 1b5, 1b6, 1b7, 8a, 8b, 8c, 8d, 9a1, 9b1, 11a, 11b, 11c, 11d, 16, 19 from a release state to a connection state and from the connection state to the release state.

The base unit 1a can comprise a device chassis in which further assemblies and components can be accommodated, as shown in FIG. 1. As part of the number of connection units 1b4, 1b5, 1b6, 1b7, 8a, 8b, 8c, 8d, 9a1, 9a2, 9b1, 11a, 11b, 11c, 11d, 16, 18, 19, a number of connection units 8a, 8b, 8c, 8d provided on the device side is provided on the base unit 1a. In all the embodiments shown, the number of connection units 8a, 8b, 8c, 8d provided on the device side is a plurality, namely four connection units 8a, 8b, 8c, 8d provided on the device side, although this is not necessary. The connection units 8a, 8b, 8c, 8d provided on the device side can be of essentially any design.

The housing unit 1b can be configured as a housing cover 1b, as shown, although this is not necessary. The housing unit 1b can be configured in several parts or in one part. In the illustrated embodiment example, the housing unit 1b comprises the housing elements 1b1, 1b2, 1b3.

The user interface 2 can be configured in one or more parts. In the embodiment example shown, the user interface 2 comprises a cover plate 2a with a control unit 2b attached to it, such as a display 2b. The cover plate 2a and the control unit 2b can be connected to each other via a mechanism or a joint, so that the control unit 2b can be pivotable relative to the cover plate 2a, for example.

It is possible that the coupling device 9a, 9b is configured in several parts.

It is preferred and can be seen in FIG. 1 that the user interface 2, may comprise as (i.e. another) part of the number of connection units 1b4, 1b5, 1b6, 1b7, 8a, 8b, 8c, 8d, 9a1, 9a2, 9b1, 11a, 11b, 11c, 11d, 16, 18, 19 a number of connection units 11a, 11b, 11c, 11d provided on the user interface side for reversibly connecting the user interface 2 to the base unit 1a, wherein the number of connection units 11a, 11b, 11c, 11d provided on the user interface side for connecting the user interface 2 to the base unit 1a can be positively connectable to the number of connection units 8a, 8b, 8c, 8d provided on the device side. The number of connection units 11a, 11b, 11c, 11d provided on the user interface side can be essentially any number.

In the embodiment example shown, the number of connection units 11a, 11b, 11c, 11d provided on the user interface side corresponds to the number of connection units 8a, 8b, 8c, 8d provided on the device side, although this is not necessary.

It is preferred and can be seen in FIG. 1 that the housing unit 1b has, as (i.e. yet another) part of the connection units 1b4, 1b5, 1b6, 1b7, 8a, 8b, 8c, 8d, 9a1, 9a2, 9b1, 11a, 11b, 11c, 11d, 16, 18, 19, a number of connection units 1b4, 1b5, 1b6, 1b7 provided on the housing unit side for reversibly connecting the housing unit 1b to the base unit 1a. In the illustrated embodiment example, the number of connection units 1b4, 1b5, 1b6, 1b7 provided on the housing unit side corresponds to the number of connection units 8a, 8b, 8c, 8d provided on the device side, although this is not necessary. The number of connection units 1b4, 1b5, 1b6, 1b7 provided on the housing unit side can essentially be configured as desired.

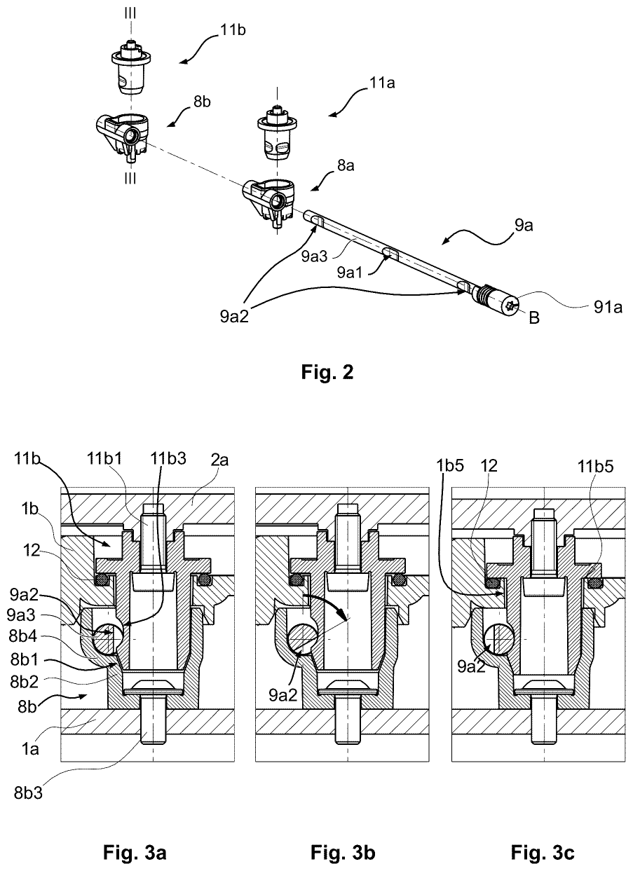

FIG. 2 shows a detailed view of a coupling device 9a, 9b according to the invention, a number of connection units 8a, 8b provided on the device side and a number of connection units 11a, 11b provided on the user interface side. These can be part of the medical device 100 explained in connection with FIG. 1 or form a separate embodiment.

In FIG. 2, only a part of the coupling device 9a, 9b is shown, since the coupling device 9a, 9b in the embodiment example according to FIG. 1 and FIG. 2 comprises two essentially identical assemblies 9a, 9b, so that what is said for the part 9a applies accordingly to the part 9b not shown in FIG. 2 and vice versa.

The coupling device 9a, 9b can have a number of locking shafts 9a3. In the embodiment example according to FIG. 1, these can be two locking shafts, of which the locking shaft 9a3 is shown in detail in FIG. 2.

Each of the number of locking shafts 9a3 can be rotatable about a respective central axis B, so that the coupling device 9a can be actuated, i.e. by rotating the locking shaft 9a3.

It is preferred, and also shown in FIG. 1 and FIG. 2, that the coupling device 9a, 9b has a number of actuators 91a, 91b to actuate the coupling device 9a, 9b, wherein this number of actuators 91a, 91b cannot be actuated without a tool. In FIG. 2, it can be seen in this respect that the actuator 91a of the number of actuators 91a, 91b can be actuated with a tool 17, such as an external hexalobular screwdriver.

The locking shaft 9a3 can be rotatably mounted in some or all of the connecting units 8a, 8b, 8c, 8d provided on the device side. In the embodiment example according to FIG. 2, both the connection unit 8a provided on the device side and the connection unit 8b provided on the device side have a corresponding bearing element (without reference signs in FIG. 2), so that two connection units 8a, 8b of the medical device 100 provided on the device side jointly accommodate a locking shaft 9a3. However, this is not necessary.

Each locking shaft can have one or more sections with recesses, whereby each recess of a locking shaft is referred to as a shaft recess. In the illustrated embodiment example according to FIG. 2, the locking shaft 9a3 has a total of three shaft recesses 9a1, 9a2. In the assembled state, the two shaft recesses 9a2 are accommodated in a respective connecting unit 8a, 8b provided on the device side.

Each of the number of connection units 11a, 11b, 11c, 11d provided on the user interface side can be configured as a connection pin, as can be seen in FIG. 2. Each of the number of connection units 11a, 11b, 11c, 11d provided on the user interface side can have a connection recess 11b3 (shown in FIG. 2 without reference characters), for example configured as a hollow groove.

FIGS. 3a to 3c show a sectional view of the detailed view according to FIG. 2 in a sectional plane in which line III-III in FIG. 2 lies and on which the center axis B is perpendicular, in an assembled state and in a state in which the base unit 1a, the housing unit 1b, and the user interface 2 are assembled.

FIGS. 3a to 3c show by way of example that each of the number of connection units 8b provided on the device side can be attached to the base unit 1a by means of a fastener such as a screw 8b3. Similarly, each of the connection units 11b provided on the user interface side can be attached to the user interface 2, for example to its cover plate 2a, by means of a fastener such as a screw 11b1. In both cases, it is possible to provide an integral configuration with the base unit 1a or with the user interface 2 instead of the respective fastening by means of a fastener.

While details of the invention are shown in FIGS. 3a to 3c only for one of the number of connection units 8b provided on the device side, for one of the connection units 11b provided on the user interface side and for one of the connection units 1b5 provided on the housing unit side, all other corresponding units can be configured in the same way or in mirror image, so that a repetitive reproduction is dispensed with.

In FIG. 3a, the base unit 1a, the housing unit 1b and the user interface 2 are assembled via their respective connection units 8b, 11b and 1b5, but not yet connected, as the coupling device 9a with the locking shaft 9a3 has not yet been actuated to provide the connection state.

In the example shown, the connecting unit 8b provided on the device side is configured as a hollow body, which can comprise a sleeve 8b2 with a pre-described position element 8b4 formed therein or thereon. The inside of the connecting unit 8b provided on the device side can also have a cone 8b1. As has already been described, the bearing element 8b4 accommodates the locking shaft 9a3, so that the shaft recess 9a2 formed in the locking shaft 9a3 is arranged in the region of the connecting unit 11b provided on the user interface side. In the assembled state, the connecting unit 11b provided on the user interface side is arranged in alignment with the connecting unit 8b provided on the device side and can essentially be configured as a pin-shaped connecting pin 11b, which can be brought into positive engagement with the connecting unit 8b provided on the device side. If the connection unit 8b provided on the device side comprises a cone 8b1 as shown, it is preferred that the connection unit 11b provided on the user interface side has a corresponding chamfer or edge in order to come into contact with the cone 8b1 in a centering manner. The connection unit 11b provided on the user interface side can have a contact surface 11b5 in order to clamp the housing unit 1b between the base unit 1a and the user interface 2. As a connection unit 1b5 provided on the housing unit side, the housing unit 1b can have, for example, a through hole 1b5, as shown, through which the connection units 11b provided on the user interface side can be guided in order to restrict the mobility of the housing unit 1b in a horizontal direction with respect to the viewing plane. Further and/or other, e.g. form-fit, pairings of the housing unit 1b with the user interface 2 and/or with the base unit 1a and/or with the connection unit 8b provided on the device side are possible.

It is possible and shown in FIGS. 3a to 3c that each locking shaft 9a3 of the number of locking shafts 9a3 can have a section with a shaft recess 9a2.

As shown in FIGS. 3a to 3c, each locking shaft 9a3 of the plurality of locking shafts 9a3 can be configured such that by rotating the locking shaft 9a3, the shaft recess 9a2 can be brought into alignment with a connection recess 11b3 of the plurality of connection recesses 11b3 to provide the release state. A correspondingly achieved release state is shown in FIG. 3a, in which the user interface 2 can be removed in the vertical direction with respect to the viewing plane, since the shaft recess 9a2 is aligned towards the connection recess 11b3.

As also shown in FIGS. 3a to 3c, each locking shaft 9a3 of the number of locking shafts 9a3 can be configured so that the shaft recess 9a2 cannot be brought into alignment with the connection recess 11b3 of the number of connection recesses 11b3 by rotating the locking shaft 9a3 in order to provide the connection state. FIG. 3b shows an intermediate state that can be achieved by rotating the locking shaft 9a3 along the direction of rotation indicated by the arrow in FIG. 3b, so that the locking shaft 9a2 partially engages with the connection unit 11b provided on the user interface side. FIG. 3c shows the connection state that can be achieved by further rotation, in which the shaft recess 9a2 is not aligned with the connection recess 11b3, so that the user interface 2 cannot be removed from the base unit 1a. During the rotation of the locking shaft 9a3, the housing unit 1b is locked between the connection unit 8b provided on the device side and the connection unit 11b provided on the user interface side by means of the connection unit 1b5 provided on the housing unit side, in the example shown by means of the through hole 1b5.

In other words, the number of connection units 1b5 provided on the housing unit side is configured to be positively connected in the connection state to the number of connection units 8b provided on the device side and/or positively connected to the number of connection units 11b provided on the user interface side.

Depending on the direction of rotation of the locking shaft 9a3, a rotation of the locking shaft 9a3 not only leads to locking, but also to a jamming of the connection unit 11b provided on the user interface side in the connection unit 8b provided on the device side. This advantageous effect occurs using the direction of rotation shown in FIG. 3b.

According to the preferred embodiment of the medical device 100 according to the invention described above, an almost play-free (backlash-free) connection is advantageously created.

In this context, it is particularly preferred and shown in FIGS. 3a to 3c that at least one sealing element 12 is arranged between the part or all of the number of contact surfaces 11b5 and the number of connection units 8b provided on the device side, which sealing element 12 is arranged and configured to exhibit a preload by producing the clamping receptacle. For example, the sealing element 12 can be arranged between some or all of the number of contact surfaces 11b5 and some or all of the number of connection units 1b5 provided on the housing unit side. It can be seen that the sealing element 12, which is not substantially prestressed in the release state, is prestressed, i.e. elastically compressed, in the connection state by clamping the number of connection element units 11b provided on the user interface side in the number of connection units 8b provided on the device side. As a result, the medical device 100 can be sealed particularly favorably on the inside.

If the medical device 100 is configured as described above, it is preferably possible to remove or attach the housing unit 1b and remove or attach the operating part 2 along a common axis A, preferably along a vertical axis A of the base unit 1a, in the release state. The common axis A or the vertical axis A corresponds to the axis A shown as an example in FIG. 1.

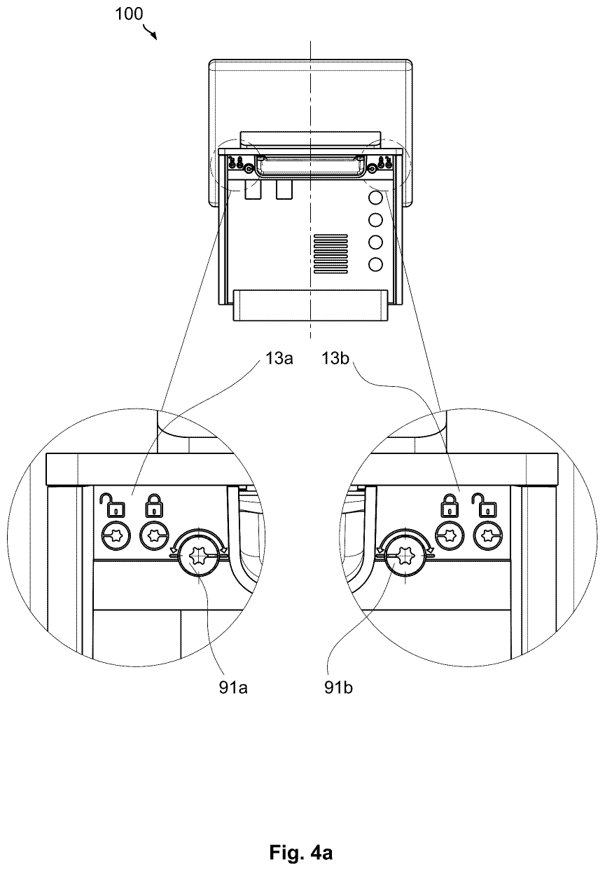

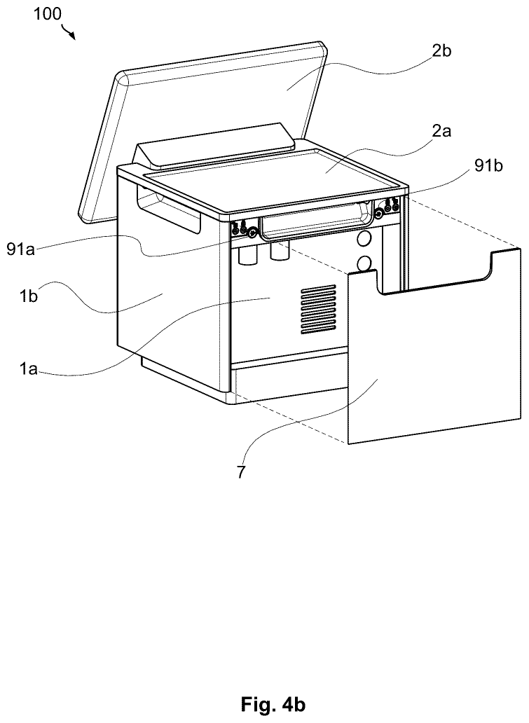

FIG. 4a shows that the number of actuators 91a, 91b in the medical device 100 according to the invention as shown in FIG. 1 can be accessible from a rear side of the medical device 100. It is preferred that pictograms 13a, 13b indicate with which actuating action the coupling device 9a, 9b can be actuated and furthermore which state of the actuators 91a, 91b corresponds to the release state or the connection state.

FIG. 4b shows that the medical device 100 according to FIG. 1 can preferably have an openable cover 7, which can be configured to conceal the number of actuators 91a, 91b in the closed state. In the embodiment example shown in FIG. 4b, the openable cover 7 is configured as a completely removable housing element 7.

If the number of connection units 8a, 8b, 8c, 8d provided on the device side and the coupling device 9a, 9b, as is the case in FIG. 1, are arranged in mirror image with respect to a vertical plane or elevated plane of the medical device 100, the coupling device 9a, 9b may require different actuating actions to provide the release state or connection state, so that the pictograms shown in FIG. 4a take this circumstance into account.

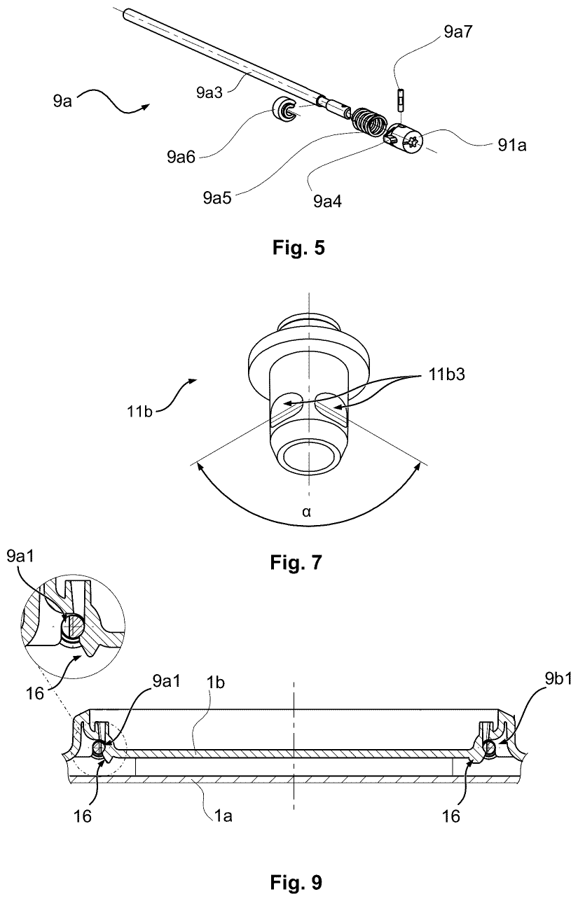

It is preferred that the medical device 100 according to FIG. 1 also has a locking device which can be configured to lock the coupling device 9a, 9b in the connection state. A corresponding embodiment example with a modified connecting shaft 9a3 is shown in perspective view in FIG. 5. If several locking shafts 9a3 are provided, each locking shaft 9a3, 9b3 can be configured accordingly.

The locking shaft 9a3 or the actuator 91a can have a stop projection 9a4. Furthermore, the locking shaft 9a3 can have a compression spring 9a5. Furthermore, the locking shaft 9a3 can have a pin 9a7. Furthermore, the locking shaft 9a3 can have a stop ring 9a6, which can be formed integrally with the locking shaft 9a3 as part of the latter or which can be provided as a separate component on the locking shaft 9a3. The stop ring 9a6 can, for example, be supported on the locking shaft 9a3 or on the bearing element 8b4 of the connection unit 8b provided on the device side. However, the stop ring 9a6 can also be dispensed with completely.

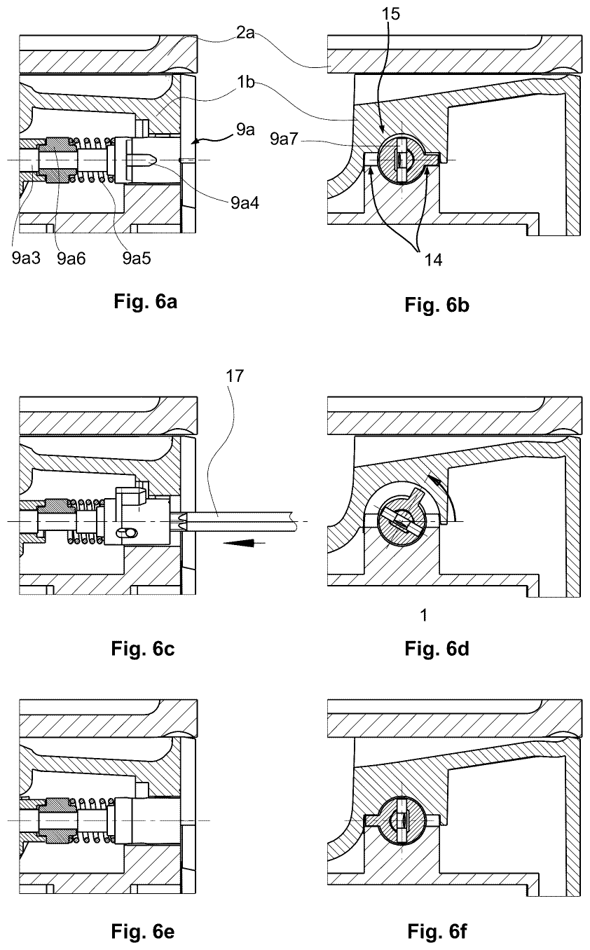

The locking shaft 9a3, the stop ring 9a6, the compression spring 9a5 and the locking actuation unit 91a with the stop projection 9a4 can be joined together as shown in FIGS. 5 and 6a to 6f by means of a pin 9a7, in that the compression spring 9a6 and the locking actuation unit 91a are pushed onto the locking shaft 9a3 and then secured with the pin 9a7, which is pressed into the slotted transverse bores of the locking actuation unit 91a and the locking shaft 9a3. In this way, the compression spring 9a5 is held in a preloaded position. When the base unit 1a is open, this assembly can simply be pushed into the corresponding connection unit 8a, 8b of the number of connection units 8a, 8b provided on the device side up to a preferably provided stop, which is advantageous for assembly and service.

FIGS. 6a to 6f show broken-out sectional views of a modified locking shaft 9a3 described above in the installed state in side view. In the installed state, the stop projection 9a4 is received between locking ribs 14 of the base unit 1a and locking ribs 15 of the housing unit 1b. Depending on the design of the locking ribs 14, 15 and the stop projection 9a4, the angular range in which the locking shaft 9a3 can rotate is limited. In the illustrated embodiment example, the achievable angular range is approx. 180°, for example. It is preferable that there are two locking states so that the locking shaft 9a3 can be locked both in the connection state and in the release state. This can be achieved, for example, by two recesses in which the stop projection 9a4 can be locked. The recesses can be formed by the locking ribs 14, 15. In order to release the locking shaft 9a3 from the locking device or from the locking devices, it is preferably necessary to actuate a locking actuation unit 91a of the locking device to actuate the locking device, wherein the locking actuation unit 91a requires an actuation action for actuation, which is different from an actuation action for actuating the coupling device 9a. This is achieved in the embodiment example according to FIGS. 6a to 6f in that the compression spring 9a5 provides a pretension of the locking shaft 9a3, which requires a displacement of the locking actuation unit 91a on the locking shaft 9a3 axially along the central axis B, preferably by means of a tool 17, in order to release the stop projection 9a4 from its corresponding recess, i.e. from the locking position, and thus to be able to rotate the locking shaft 9a3. A corresponding process is shown sequentially in FIGS. 6a to 6f.

FIGS. 6a and 6b show the release state of the coupling device 9a, 9b, in which the user interface 2 and the housing unit 1b can be removed from or attached to the base unit 1a. The locking ribs 14, 15 catch the stop projection 9a4 from above and below, so that the open angular position of the locking shaft 9a3 is fixed, i.e. locked, and thus a rotary movement is initially not possible.

FIGS. 6c and 6d show the locking process: the locking actuating unit 91a must be pressed in against the restoring force of the compression spring 9a5 when driven with a tool 17 and is guided longitudinally on the locking shaft 9a3 so that the stop projection 9a4 comes free against the locking ribs 15. The rotary movement is only possible in this position. In contrast, the locking ribs 14 also remain engaged in this plane of rotation of the stop projection 9a4, so that a rotary movement of the locking shaft 9a3 is only possible in the intended angular range of 180°, for example.

FIGS. 6e and 6f show the locked state and the connection state in which the user interface 2 and the housing unit 1b are connected to the base unit 1a. Axial release of the locking actuation unit 91a causes it to move back into the axial starting position due to the spring force of the compression spring 9a5, so that the locking ribs 14, 15 again catch the stop projection 9a4 from above and below. This fixes the locking shaft 9a3 in the locked position, i.e. locks it in place, so that unintentional rotation is not possible.

FIG. 9 shows a broken-out sectional view through a plateau or vertical plane of a further embodiment of a medical device 100.

FIG. 9 shows that the medical device 100 can have a number of additional connection units 9a1, 9b1 provided on the device side for reversibly connecting the base unit 1a to the housing unit 1b without the user interface 2 having to be connected to the base unit 1a. In the illustrated embodiment example, this is realized in that at least one of the locking shafts 9a3, 9b3—in the illustrated embodiment example, each of the locking shafts 9a3, 9b3—has a further shaft recess 9a1, 9b1. Furthermore, it is shown in the illustrated embodiment example that the housing unit 1b can have a further number of connection units 16 provided on the housing element side for connection to the number of additional connection units 9a1, 9b1 provided on the device side. In the illustrated embodiment example, the housing unit 1b has two further connection units 16 provided on the housing element side for this purpose, although this is not necessary. The further number of connection units 16 provided on the housing element side can, for example, be configured as a number of hooks 16, which can be brought into positive engagement with the number of additional connection units 9a1, 9b1 provided on the device side. By placing the housing unit 1b on the base unit 1a and correspondingly actuating the number of additional connection units 9a1, 9b1 provided on the device side, in the illustrated embodiment example achievable by rotating the number of locking shafts 9a3, 9b3, a connection between the base unit 1a and the housing unit 1b can be achieved. Such a procedure offers advantages for device logistics.

FIG. 1 also shows that it is preferable for the base unit 1a to have one or more projections 18 in order to provide a reversible connection with the housing element 1b. For this purpose, the housing element 1b can have an additional number of connection units 19 provided on the housing element side. The one or more projections 18 can, for example, be configured as a plurality of catch teeth 18. The additional number of connecting units 19 provided on the housing element side may, for example, be substantially pocket-like in design such that the additional number of connecting units 19 provided on the housing element side come into engagement with the one or more projections 18 when the housing unit 1b is placed on the base unit 1a. It is preferred that the one or more projections 18 and/or the preferably pocket-like additional number of connecting units 19 provided on the housing element side are each provided with insertion chamfers in order to facilitate placement. It is particularly advantageous to provide a plurality of projections 18 in the form of catch teeth, which are not all the same length, so that not all catch teeth 18 engage with the additional number of connecting units 19 provided on the housing element side at the same time. This makes assembly easier.

FIG. 1 shows that it is preferred that the number of connection units 8a, 8b, 8c, 8d provided on the device side is configured as a plurality, with the plurality of connection units 8a, 8b, 8c, 8d provided on the device side being distributed evenly around the common axis A in order to provide a plurality of arrangements of the user interface 2 and/or the housing unit 1b relative to the base unit 1a. In the illustrated embodiment example, for example, four orientations of the user interface 2 relative to the base unit 1a can be achieved by rotating the user interface 2 accordingly about the common axis A. In this context, it is preferred that, if each of the number of connection units 11a, 11b, 11c, 11d provided on the user interface side is essentially configured as a pin-shaped connection pin 11b as described above, the connection pin 11b has a plurality of connection recesses 11b3, which can be brought into engagement with the respective locking shaft 9a3 in the manner described above, depending on the orientation of the user interface 2. The number of the plurality of connection recesses 11b3, together with the number of connection units 11a, 11b, 11c, 11d provided on the user interface side, determines the possible number of orientations of the user interface 2 relative to the base unit 1a. In the illustrated embodiment example according to FIG. 7, it is preferred that the plurality of connection recesses 11b3 are evenly distributed around a central axis of the connection pin 11b, with two adjacent connection recesses 11b3 enclosing a common predetermined angle α in each case. The common predetermined angle α in the illustrated embodiment example is essentially 90 degrees, for example, but may deviate from this.

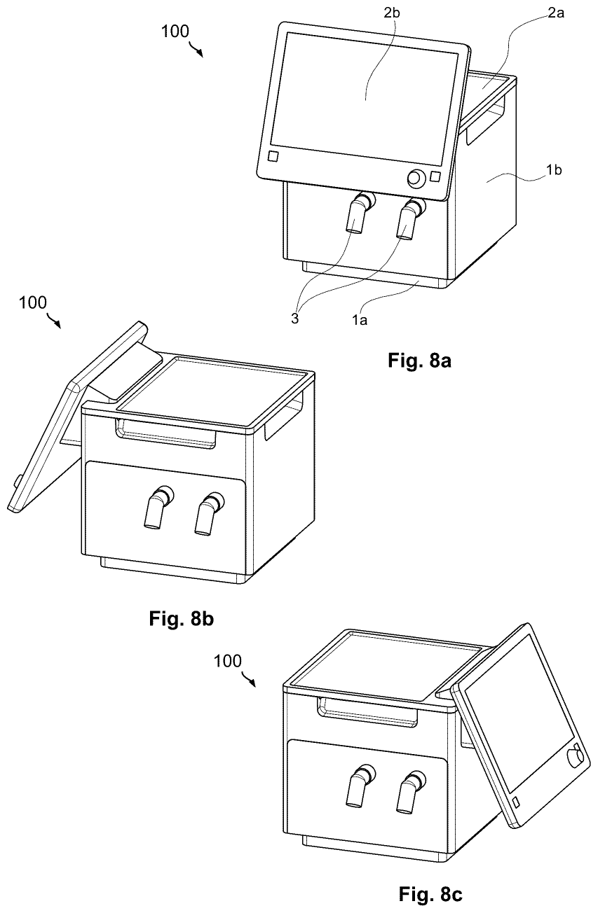

FIGS. 8a, 8b, 8c show three different embodiments of a medical device 100 according to the invention, which can be achieved by arranging the user interface 2 and/or the housing unit 1b in different ways relative to the base unit 1a.

FIG. 8a shows a medical device 100 in which the user interface 2 is oriented in the same direction as the patient connections 3.

FIG. 8b shows a medical device 100 in which the user interface 2 is arranged to the left of the patient connections 3 in the viewing direction.

FIG. 8c shows the medical device 100 in which the user interface 2 is arranged in the viewing plane and to the right of the patient connections 3. The arrangement of the user interface 2 to the left or right of the patient connections 3 offers the possibility of connecting the control unit 2b of the user interface 2, if present, to the cover plate 2a, if present, in a lower position than in the arrangement shown in FIG. 8a, which enables a more compact design.

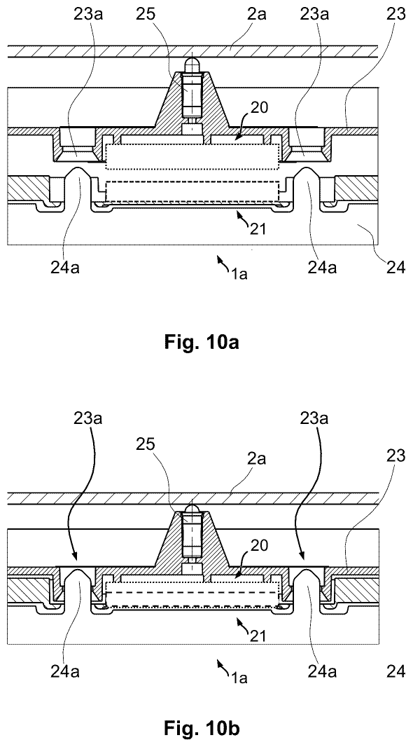

It is preferred and shown in FIG. 10a and FIG. 10b that an automatic electrical and/or signal connection between the user interface 2 and the base unit 1a can be achieved when the medical device 100 is mounted. For this purpose, it is preferred that the user interface 2 has a first electrical and/or signaling interface 20, wherein the base unit 1a has a corresponding second electrical and/or signaling interface 21, wherein the first interface 20 and the second interface 21 can be connected to establish an electrical and/or signaling connection.

In the embodiment example shown, the user interface 2 or its cover plate 2a has a contact base 23, whereby the first interface 20 is configured as a connector, which is floatingly mounted in or on the user interface 2, in that the contact base 23 is floatingly mounted on the cover plate 2a. In the example shown, the second interface 21 is permanently installed on a printed circuit board 24, which is connected to the base unit 1a. It is preferred and shown in FIGS. 10a, 10b that the user interface 2 and/or the base unit 1a and/or the housing element 1b has a centering device 23a, 24a in order to align the first interface 20 and the second interface 21 with one another in at least one direction when the user interface 2 is placed on the base unit 1a. In the illustrated embodiment example, the printed circuit board 24 has lugs 24a for this purpose, which can be brought into engagement with catch funnels 23a of the user interface 2.

It is also preferred and shown in FIGS. 10a, 10b that the connector 20 is spring-loaded in order to provide a contact pressure on the connector 20 in the direction of the second interface 21. In the illustrated embodiment example, this is achieved by a spring-loaded pressure piece 25, which can be braced between the cover plate 2a and the contact base 23.

It is further preferred that an orientation of the first interface 20 relative to the user interface 2 and/or an orientation of the second interface 21 relative to the base unit 1a is variable. In this way, it is possible for the first interface 20 and the second interface 21 to be connectable at any possible orientation of the user interface 2 relative to the base unit 1a.

All of the features disclosed herein can be combined with each other, unless this concerns alternatives or is contradictory.

While specific embodiments of the invention have been shown and described in detail to illustrate the application of the principles of the invention, it will be understood that the invention may be embodied otherwise without departing from such principles.

### List of Reference Characters

1a Base unit
1b Housing unit
1b1, 1b2, 1b3 Housing element
1b4, 1b5, 1b6, 1b7 Connection unit provided on the housing unit side, through hole
2 User interface
2a User interface cover plate
2b Control unit, user interface display
3 Patient connections
7 Cover
8a, 8b, 8c, 8d Connection unit provided on the device side
8b1 Cone
8b2 Sleeve
8b3 Screw
8b4 Bearing element
9a, 9b Coupling device
9a1, 9b1 Additional connection unit provided on the device side, additional shaft recess
9a2 Shaft recess
9a3 Locking shaft
9a4 Stop projection
9a5 Compression spring
9a6 Stop ring
9a7 Pin
11a, 11b, 11c, 11d Connection unit provided on the user interface side, connection pin
11b1 Screw
11b3 Connection recess, fillet
11b5 Contact surface
12 Sealing element
13a, 13b Pictogram
14 Locking ribs on the base unit
15 Locking ribs on the housing unit
16 Additional number of connection units provided on the housing element side
17 Tool
18 Projection
19 Additional number of connection units provided on the housing element side
20 First interface, connector
21 Second interface
23 Contact base
23a Centering device, catching funnel
24 Circuit board
24a Centering device, nose (lug)
25 Spring-loaded thrust piece
91a, 91b Actuators, locking actuating unit
100 Medical device
A Common axis, vertical axis
B Center axis of the locking shaft
α Predetermined angle

What is claimed is:

1. A medical device comprising:
a base unit:
a housing unit, which is configured to be connected to the base unit;
a user interface, which is configured to be connected to the base unit;
a number of connection units configured for reversibly connecting the base unit to the housing unit and for reversibly connecting the base unit to the user interface; and
a coupling device, which is configured to change the number of connection units from a release state to a connection state and from the connection state to the release state.

2. A medical device according to claim 1, wherein the coupling device comprises a number of actuators configured for actuating the coupling device, wherein the actuators cannot be actuated without tools.

3. A medical device according to claim 2, further comprising an openable cover, which is arranged in a closed state to conceal the number of actuators.

4. A medical device according to claim 1, wherein the coupling device comprises a number of locking shafts, each of the number of locking shafts being rotatable about a respective center axis for operating the coupling device.

5. A medical device according to claim 1,
wherein a part of the number of connection units is configured as a number of connection units provided on a device side for reversibly connecting the base unit to the housing unit and for reversibly connecting the base unit to the user interface,
wherein a part of the number of connection units is configured as a number of connection units provided on a user interface side for reversibly connecting the user interface to the base unit, and
wherein the number of connection units provided on the user interface side for connecting the user interface to the base unit can be positively connected to the number of connection units provided on the device side.

6. A medical device according to claim 5, wherein the number of connection units provided on the device side is configured to enable removal or attachment of the housing unit and removal or attachment of the user interface along a common axis, in the release state.

7. A medical device according to claim 6,
wherein the number of connection units provided on the device side is configured as a plurality of connection units provided on the device side, and
wherein the plurality of connection units provided on the device side is evenly distributed around a common axis to provide a plurality of arrangements of the user interface and/or the housing unit relative to the base unit.

8. A medical device according to claim 6,
wherein the coupling device comprises a number of locking shafts, each of the number of locking shafts being rotatable about a respective center axis for operating the coupling device,
wherein each connection unit of the number of connection units provided on the user interface side has a connection recess,
wherein each locking shaft of the number of locking shafts has a section with a shaft recess,
wherein each locking shaft of the number of locking shafts is configured such that, by rotating the locking shaft, the shaft recess is configured to be brought into alignment with one of the connection recesses of the number of connection units to provide the release state, and
wherein each locking shaft of the number of locking shafts is configured such that by rotating the locking shaft, the shaft recess is configured to not be in alignment with the connection recess of the number of connection units to provide the connection state.

9. A medical device according to claim 7, wherein a part of the number of connection units provided on the device side comprises a number of connection units provided on a housing unit side for reversibly connecting the housing unit to the base unit.

10. A medical device according to claim 8, wherein a part of the number of connection units provided on the device side comprises a number of connection units provided on a housing unit side for reversibly connecting the housing unit to the base unit.

11. A medical device according to claim 9, wherein the number of connection units provided on the housing unit side is configured to be positively connected in the connection state to the number of connection units provided on the device side and/or positively connected to the number of connection units provided on the user interface side.

12. A medical device according to claim 10, wherein the number of connection units provided on the housing unit side is configured to be positively connected in the connection state to the number of connection units provided on the device side and/or positively connected to the number of connection units provided on the user interface side.

13. A medical device according to claim 8,
wherein at least a part of the number of connection units provided on the user interface side has a respective contact surface for clampingly receiving the housing unit between the user interface and the base unit,
wherein the clamping reception is achieved by providing the connection state by rotating the number of locking shafts.

14. A medical device according to claim 13, further comprising at least one sealing element, which is arranged between one or all of the number of contact surfaces and the number of connection units provided on the device side, which sealing element is configured to exhibit a prestress by producing the clamping receptacle.

15. A medical device according to claim 1, further comprising a locking device which is arranged to lock the coupling device in the connection state.

16. A medical device according to claim 15,
wherein the locking device comprising a locking actuating unit for actuating the locking device,
wherein the locking actuating unit requires an actuating action for actuation which is different from an actuating action for actuating the coupling device.

17. A medical device according to claim 1, further comprising a number of additional connection units provided on the device side for reversibly connecting the base unit to the housing unit without the user interface being connected to the base unit.

18. A medical device according to claim 1,
wherein the user interface comprises a first electrical and/or signaling interface,
wherein the base unit comprises a corresponding second electrical and/or signaling interface, and
wherein the first interface and the second interface are configured to be connected to establish an electrical and/or signaling connection.

19. A medical device according to claim 18, wherein an orientation of the first interface relative to the user interface and/or an orientation of the second interface relative to the base unit is variable.

20. A medical device according to claim 18,
wherein the first interface is configured as a connector which is floatingly mounted in or on the user interface, and
wherein the connector is spring-loaded to provide a contact pressure on the connector in a direction of the second interface.
